(19) United States
(12) Patent Application Publication
(10) Pub. No.: US 2024/0058308 A1
(43) Pub. Date: Feb. 22, 2024

(54) TREATMENT AND PREVENTION OF DRY MACULAR DEGENERATION

(71) Applicant: The Regents of the University of Michigan, Ann Arbor, MI (US)

(72) Inventor: Jason Miller, Ann Arbor, MI (US)

(21) Appl. No.: 18/260,919

(22) PCT Filed: Jan. 12, 2022
(86) PCT No.: PCT/US22/12173

§ 371 Date: Jul. 10, 2023

Related U.S. Application Data
(60) Provisional application No. 63/136,334, filed on Jan. 12, 2021.

(51) Int. Cl. A61K 31/4184; A61P 27/02

(52) U.S. Cl. CPC A61K 31/4184; A61P 27/02

(57) ABSTRACT

Provided herein are methods for the treatment and prevention of dry macular degeneration via the pharmacologic activation of autophagy without direct inhibition of mammalian target of rapamycin (mTOR), for example by administration of flubendazole.

### CROSS-REFERENCE TO RELATED APPLICATIONS

The present invention claims priority to U.S. Provisional Patent Application Ser. No. 63/136,334, filed Jan. 12, 2021, which is incorporated by reference in its entirety.

### STATEMENT REGARDING FEDERAL FUNDING

This invention was made with government support under EY007003 awarded by the National Institutes of Health. The government has certain rights in the invention.

### FIELD

Provided herein are methods for the treatment and prevention of dry macular degeneration via the pharmacologic activation of autophagy without direct inhibition of mammalian target of rapamycin (mTOR), for example by administration of flubendazole (FLBZ).

### BACKGROUND

Age-related macular degeneration (AMD) is the leading cause of blindness in the developed world. While the wet form, characterized by fibrovascular scarring, can be prevented with anti-VEGF agents, there are no therapies for the dry form of the disease, which affect 90% of AMD patients. Dry age-related macular degeneration (dAMD) is marked by the accumulation of extracellular and intracellular lipid-rich debris within and around the retinal pigment epithelium (RPE).

### SUMMARY

Provided herein are methods for the treatment and prevention of dry macular degeneration via the pharmacologic activation of autophagy without direct inhibition of mammalian target of rapamycin (mTOR), for example by administration of flubendazole.

In some embodiments, provided herein are methods of treating or preventing dry age-related macular degeneration (dAMD) in a subject comprising the induction of autophagy in the retinal pigment epithelium (RPE) of the subject without direct inhibition of mammalian target of rapamycin (mTOR). In some embodiments, autophagy is induced in the RPE by administration of a pharmacologic activator of autophagy to the RPE that does not directly inhibit mTOR. In some embodiments, the pharmacologic activator of autophagy does not bind or otherwise directly inhibit mTOR or its immediately upstream kinases, AKT and the PI3K family. In some embodiments, the pharmacologic activator of autophagy is administered directly to the eye of the subject. In some embodiments, the pharmacologic activator of autophagy is flubendazole. In some embodiments, the subject exhibits one or more risk factors of developing dAMD or exhibits early signs or symptoms of dAMD.

In some embodiments, a method comprises (a) testing a subject for dAMD; and (b) treating a subject that exhibits signs or symptoms of dAMD with the method herein. In some embodiments, testing the subject for dAMD comprises one or more of an eye exam; review of medical history, review of family medical history, examination of the back of the eye, testing for defects in the center of the field of vision, angiography, and optical coherence tomography. In some embodiments, a subject is determined to be at risk of dAMD and/or exhibits early signs or symptoms of dAMD and the subject is treated to prevent development and/or advancement of dAMD.

In some embodiments, provided herein are pharmaceutical compositions comprising (a) a pharmacologic activator of autophagy that does not directly inhibit mammalian target of rapamycin (mTOR) and (b) a pharmaceutically acceptable carrier. In some embodiments, the pharmacologic activator of autophagy is flubendazole. In some embodiments, the pharmaceutical composition is formulated for delivery to the RPE.

### BRIEF DESCRIPTION OF THE DRAWINGS

FIG. 1. Schematic of the choroid, retinal pigment epithelium (RPE), and photoreceptors of a healthy eye (top) and an eye of a subject suffering from dry age-related macular degeneration (bottom). Formation of drusen is the hallmark of dAMD.

FIG. 2. Identification of autophagy inducers in primary human fetal (hfRPE) culture. (A) Induction of autophagy by analysis of LC3 lipidation (LC3-II/LC3-I ratio). Cultures were exposed to compounds or vehicle (DMSO) for 24 hours. Torin n=8, GSK n=10, FLBZ n=10, D4476 n=8, GW7647 n=11, JNJ n=10, Amiodarone n=8. Torin and GSK are mTOR-dependent autophagy inducers, while the other compounds are mTOR-independent. (B) Induction of autophagy by analysis of LC3 puncta formation (LC3 staining in green) using LC3 Puncta Index described previously [43]. Scale bar: 10 μm. Torin n=7, GSK n=3, FLBZ n=4, D4476 n=7. (C) Autophagy flux assays. After application of inducers or vehicle (DMSO) for 22.5 hours, 25 mM of NH4Cl, a lysosomal alkalizing agent, or H2O were added for a final 1.5 hours to inhibit autophagy flux. Resulting increases in the LC3-II/LC3-I ratio indicate that the compound induces autophagy flux. Torin n=6, GSK n=3, FLBZ n=6, D4476 n=5. Uncropped blots in FIG. 8. *p<0.05, **p<0.01.

FIG. 3. Safety of confirmed autophagy inducers in primary hfRPE culture. (A) Tight junction integrity, as measured by trans-epithelial electrical resistance (TEER), is a general marker of RPE health. Drug or vehicle (DMSO) replaced daily with measurement just before drug replacement. FLBZ shows enhanced TEER while all others, especially mTOR inhibitors Torin and GSK, demonstrate progressively lower tight-junction integrity. n=6. (B) Cytotoxicity as measured by percent of total possible LDH release into the apical supernate. Drug or vehicle (DMSO) replaced daily with supernate collected just before drug replacement. All compounds demonstrated slightly lower cytotoxicity than DMSO control. Note scale break on Y-axis, indicating all conditions, including vehicle, demonstrated minimal LDH release. n=6. (C) Outer segment (OS) phagocytosis efficiency, as measured by disappearance of rhodopsin, the primary protein in OS. Direct mTOR inhibition (Torin, GSK) reduces phagocytosis efficiency, whereas D4476 and FL 650 BZ have no effect on phagocytosis. Torin n=12, GSK n=4, FLBZ n=9, D4476 n=3. ns p>0.05, *p<0.05, **p<0.01, ***p<0.001.

FIG. 4. Impact of confirmed autophagy inducers on RPE lipid metabolism. (A) Proposed model of RPE lipid handling. Lipid-rich shed OS are phagocytosed from the apical side and lipoprotein complexes are consumed from the basolateral side daily. Incomplete lipid degradation contributes to lipofuscin accumulation. With the remaining lipid load, we postulate that the RPE daily determines the balance between complete lipid degradation, as assessed by KB production, and secretion of lipid via lipoprotein particles, as assessed by APOE secretion. (B) KB production (as assessed by β-hydroxybutyrate, β-HB) in the presence of vehicle (DMSO) or confirmed autophagy inducers for 24 hours. β-HB is secreted almost exclusively into the apical supernate. Only FLBZ increased lipid degradation. Torin n=4, GSK n=7, FLBZ n=6, D4476 n=4. (C) Lipoprotein secretion (as assessed by apolipoprotein E, APOE) in the presence of vehicle (DMSO) or confirmed autophagy inducers for 24 hours. Both apical and basolateral media contain APOE. While increasing lipid degradation (B), FLBZ also decreases secretion of drusen promoting lipoprotein. Apical: Torin n=4, GSK n=5, FLBZ n=11, D4476 n=4. Basal: Torin n=4, GSK n=5, FLBZ n=9, D4476 n=3. ns p>0.05, *p<0.05, **p<0.01.

FIG. 5. Autophagy inducer FLBZ reduces accumulation of lipofuscin-like material. (A) Lipofuscin-like material, which we term undigestible autofluorescent material (UAM), accumulates in RPE after repeated feedings of photo-oxidized outer segments (oxOS). UAM granules (green). DAPI (blue). Phalloidin stain of F-actin outlining cell borders (pink). Scale bar: 10 μm. (B) Effects of FLBZ or Torin on UAM accumulation (left) and elimination (right). FLBZ or Torin is fed together with oxOS daily for 20 days in a month (left, n=5) or fed daily for 20 days in a month after completion of oxOS feedings to stimulate UAM accumulation (right, n=7). Unlike Torin, FLBZ both reduces UAM accumulation and increases UAM elimination. DMSO as vehicle control. U 673 AM normalized to DMSO condition. (C) LC3 colocalization to UAM granules in the human RPE cell line, ARPE-19, treated with FLBZ. UAM (red). LC3 (green). DAPI (blue). Arrows indicate LC3 puncta surrounding a lipofuscin granule. Scale bar: 2 μm. (D) Effects of FLBZ on UAM granule size. Compared to vehicle (DMSO), FLBZ decreases UAM granule size both during oxOS feedings (left) and after UAM buildup has already occurred (right). n=40. ns p>0.05, *p<0.05, **p<0.01, ***p<0.001.

FIG. 6. FLBZ alleviates UAM-induced senescence and tight-junction disruption. (A) (Top) FLBZ reduces senescence when fed concurrently with oxOS during UAM accumulation. (Bottom) FLBZ is unable to reverse established senescence induced by already accumulated UAM. FLBZ is fed daily together with oxOS for 20 days in a month (top) or after one month of 20 oxOS feedings to induce UAM buildup (bottom). DMSO as vehicle control. Senescence measured by β-galactosidase activity (blue). Scale bar: 50 μm. n=6. (B) While FLBZ does not reduce senescence when added to culture after UAM accumulation has already occurred, it does improve RPE cell health, as assessed by tight-junction integrity (TEER measured after 20 FLBZ feedings) (right). Left graph n=6, right graph n=12. ns p>0.05, *p<0.05, ***p<0.001.

FIG. 7. Serum and amino acid starvation induce autophagy flux in hfRPE. hfRPE incubated in Hank's Balanced Salt Solution for 6 hours with normal media as control. The lysosomal alkalinizing agent NH4Cl (25 mM) was added 1.5 hours prior to harvest. In control wells, the increase in LC3-II/LC3-I ratio after blockade of autophagy flux by NH4Cl demonstrates that hfRPE has high baseline levels of constitutive autophagy. Amino acid and serum starvation with Hank's Balanced Salt Solution induces autophagy above baseline levels. n=6. **p<0.01, ***p<0.001.

FIG. 8. Uncropped Western blots for FIG. 2. Symbols (+ or −) indicate presence or absence of NH₄Cl.

FIG. 9. mTor inhibitor Torin1 reduces hfRPE pigmentation. Whole mounted Transwells (in duplicate) are photographed. Daily feeding of oxOS and Torin together during UAM buildup resulted in reduced pigmentation. In contrast, FLBZ and vehicle (DMSO) treatment result in preserved pigmentation.

FIG. 10. Lactate dehydrogenase (LDH) assay accurately assesses cell death, as determined by exposure to the oxidant tert-butyl hydroperoxide (tBHP). Primary fetal RPE cultures were exposed to tBHP for 24 hours at a concentration known to cause partial but not complete cell death on the Transwell. There is a corresponding marked increase in LDH release. Data normalized to maximum possible LDH release as well as to the no tBHP condition.

FIG. 11. Detecting Autophagy In Vivo. Western blot shows equal capacity to detect LC3II and LC3I band when 1 eye was used per sample vs. pooling two eyes.

FIG. 12. Intravitreal Injection of Torin (DMSO as control). Torin was injected intravitreally at multiple concentrations (0.5 uM up to 500 uM) and mouse eyes were harvested at various times over a week, assaying lysates for autophagy by LC3II/I ratio. All graphs involve injection of 1 ul of 500 uM Torin (the highest concentration tested). No autophagy induction occurred with Torin, the positive control.

FIG. 13. Intravitreal Injection of Flubendazole (DMSO as control). Intravitreal injection of flubendazole at a maximum soluble concentration (500 uM delivered in 1 ul of DMSO) did not result in autophagy induction at multiple timepoints after injection, as assessed by LC3II/LC3I ratio.

FIG. 14. Effect of autophagy positive control Torin on RPE from mouse eyecups (DMSO as control). To confirm induction of autophagy in mouse eyes (if penetration barriers were removed), an ex vivo method was used. Mouse eyes were enucleated, the anterior segment vitreous, and retina were removed, leaving an eyecup with RPE/choroid exposed. The eyecups were incubated with a previously established autophagy inducer, the mTOR inhibitor Torin (1-5 uM), which induced autophagy as determined by an increased LC3II/LC3I ratio on Western blotting. RPE/Choroid eyecup was treated with 1 uM Torin1 in our previously established RPE cell culture media for 24 hours. n=4.

FIG. 15. Effect of Flubendazole on RPE from Mouse Eyecups (DMSO as control). Conditions with and without ammonium chloride (NH₄Cl) help establish high autophagy flux rates. If true autophagy flux is happening, the LC3II/LC3I ratio should increase further upon addition of NH₄Cl, as is the case here. DMSO− and FLBZ−, n=6, FLBZ+ and DMSO+ n=5. Flubendazole tested at 60 uM, lysates collected after 24 hours. Induction did occur in eyecups.

FIG. 16. Transepithelial Electrical Resistance measurements for apatinib and anlotinib, two small molecules with potential to stimulate autophagy in the RPE independent of direct mTOR inhibition. No toxicity is observed compared to Torin control.

FIG. 17. Transepithelial Electrical Resistance measurements for sunitinib, a small molecule with potential to stimulate autophagy in the RPE independent of direct mTOR inhibition. No toxicity is observed.

FIG. 18A-C. Autophagy Induction as measured by LC3-II/LC3-I Ratios on Western Blot for anlotinib, apatinib, and sunitinib in hfRPE cultures. Use of these drugs was shown to be non-toxic in FIG. 16 above. There is evidence for autophagy induction in human RPE with each of these compounds.

FIG. 19. Autophagy Induction as measured by LC3 puncta on immunocytochemistry, confirming autophagy induction in hfRPE for anlotinib, apatinib, and sunitinib.

### DEFINITIONS

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject. In some embodiments, the subject may be over the age of 50.

As used herein, the term “administration” refers to the act of giving a drug, prodrug, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the terms “treat,” “treatment,” and “treating” refer to reducing the amount or severity of a particular condition, disease state (e.g., BPH), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the terms “prevent,” “prevention,” and preventing” refer to reducing the likelihood of a particular symptom, condition, or disease state from occurring in a subject not presently experiencing or afflicted with the condition or disease state. The terms do not necessarily indicate complete or absolute prevention. For example, “prevention” refers to reducing the likelihood of a symptom, condition, or disease state occurring in a subject not presently experiencing or diagnosed with the symptom, condition, or disease state. In order to “prevent” a symptom, condition, or disease state, a composition or method need only reduce the likelihood of the symptom, condition, or disease state, not completely block any possibility thereof. “Prevention,” encompasses any administration or application of a therapeutic or technique to reduce the likelihood of a disease developing (e.g., in a mammal, including a human). Such a likelihood may be assessed for a population or for an individual.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “macular degeneration” refers to ocular diseases wherein the macula—a small and highly sensitive part of the retina responsible for detailed central vision—degenerates and/or loses functional activity. The degeneration and/or loss of functional activity may be due to any reason including, without limitation, cell death or apoptosis, decreased cell proliferation, and/or loss of normal biological function. Macular degeneration may be “wet” (i.e., exudative and/or neovascular) or dry (i.e., non-exudative, atrophic and/or non-neovascular).

As used herein, the term “subject suspected of having AMD” refers to a subject that presents one or more symptoms indicative of age-related macular degeneration, has one or more risk factors for AMD, or is being screened for AMD (e.g., during a routine physical). A subject suspected of having AMD has generally not been tested for AMD, or has not had a recent test which indicated the subject suffers from AMD. However, a “subject suspected of having AMD” encompasses an individual who has received a preliminary diagnosis but for whom a confirmatory test has not been done. A “subject suspected of having AMD” is sometimes diagnosed with AMD and is sometimes found to not have AMD.

As used herein, the term “subject diagnosed with AMD” refers to a subject who has been tested and found to have AMD. AMD may be diagnosed using any suitable method, including but not limited to, the diagnostic methods of the present invention.

As used herein, the term “subject suffering from AMD” refers to a subject who has AMD and exhibits one or more symptoms thereof. A subject suffering from AMD may or may not have received a diagnosis, and may or may not be aware of the condition.

As used herein, the term “initial diagnosis” refers to a test result of initial AMD diagnosis that reveals the presence or absence or risk of AMD. An initial diagnosis does not include information about the stage or extent of AMD.

As used herein, the term “subject at risk for AMD” refers to a subject with one or more risk factors for developing AMD. Risk factors include, but are not limited to, gender, age, genetic predisposition, environmental exposure, and lifestyle.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “instructions for administering,” and grammatical equivalents thereof, includes instructions for using the compositions contained in a kit for the treatment of conditions (e.g., providing dosing, route of administration, decision trees for treating physicians for correlating patient-specific characteristics with therapeutic courses of action).

### DETAILED DESCRIPTION

Provided herein are methods for the treatment and prevention of dry macular degeneration via the pharmacologic activation of autophagy without direct inhibition of mammalian target of rapamycin (mTOR), for example by administration of flubendazole.

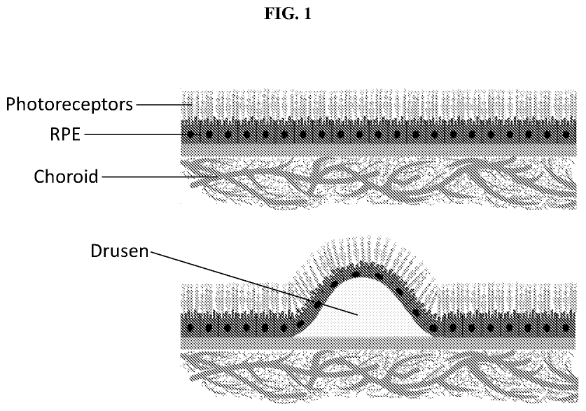

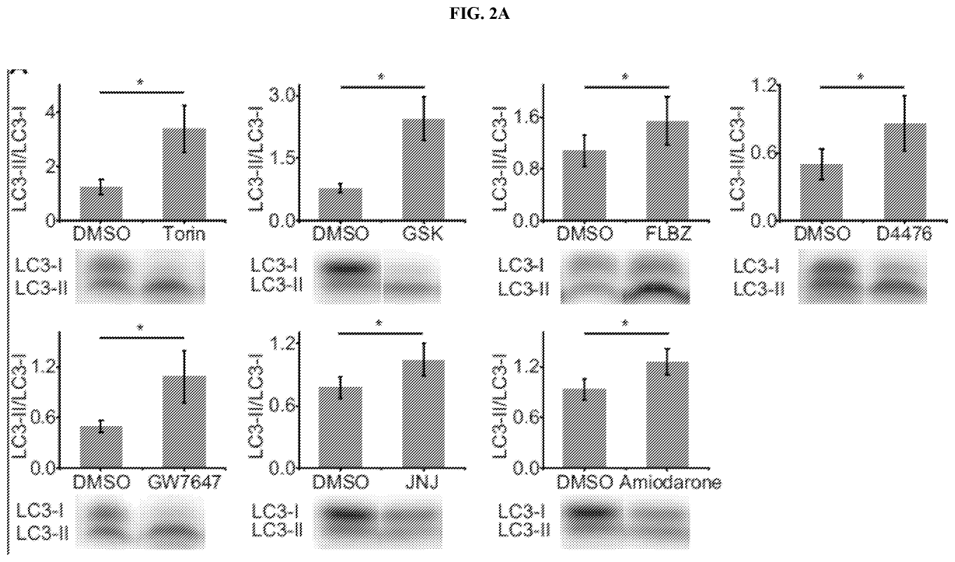

Dry age-related macular degeneration (dAMD) is marked by the accumulation of extracellular and intracellular lipid-rich debris within and around the retinal pigment epithelium (RPE). The deterioration of the retina is associated with the formation of small yellow deposits, known as “drusen,” under the RPE in the macula (FIG. 1), whitish deposits above the RPE in the macula, known as “reticular pseudodrusen” or “subretinal drusenoid deposits”, and intracellular deposits termed lipofuscin. The accumulation of these deposits leads to atrophy of the retinal pigment epithelium (RPE), a layer of the retina that is critical for macular function. As the RPE atrophies, the subject with dAMD loses central vision.

It is contemplated that inducing autophagy, a conserved, intracellular catabolic and degradative pathway, is a treatment strategy to treat/prevent dAMD by clearing these intracellular debris and decreasing the secretion of lipoprotein particles that nucleate the extracellular debris. However, mTor inhibition, the major mechanism for inducing autophagy, detrimentally alters core RPE functions. Experiments were conducted during development of embodiments herein to develop an alternative therapeutic approach, selecting over thirty putative autophagy inducers that do not act via direct mTOR inhibition. These compounds were tested for safety and efficacy in a primary human RPE culture model of AMID. Only two of the compounds reliably increased autophagy flux in the culture system in a first round of testing, emphasizing that autophagy induction mechanistically differs across distinct tissues. In contrast to direct mTOR inhibitors, these compounds preserved RPE-specific functions such as tight-junction integrity and outer segment phagocytosis. Experiments conducted during development of embodiments herein demonstrated that flubendazole (FLBZ) reduced the secretion of lipoprotein particles that generate extracellular lipid-rich debris termed drusen and reticular pseudodrusen. Simultaneously, FLBZ increased degradation of lipids, marked by the production of the lipid-degradation product β-hydroxybutyrate, which is used by photoreceptor cells as an energy source. FLBZ was also capable of reducing accumulation of intracellular lipid-rich debris, termed lipofuscin, in the RPE. Lipofuscin-like accumulation results in increased senescence and decreased tight-junction integrity in the model, which were alleviated with FLBZ treatment. Flubendazole triggered compaction of lipofuscin-like granules into a less toxic form. These results indicate that RPE autophagy induction outside of direct mTOR inhibition is a promising therapeutic approach for dry AMD.

In some embodiments, provided herein are pharmaceutical compositions comprising flubendazole, for example, in combination with a pharmaceutically acceptable carrier. In particular embodiments, the pharmaceutical composition is formulated for delivery to the eye. The composition can be formulated for local (e.g., ocular, intraocular, etc.), parenteral, oral, or topical administration. For example, a parenteral formulation could consist of a prompt or sustained release liquid preparation, dry powder, emulsion, suspension, or any other standard formulation. An oral formulation of the pharmaceutical composition could be, for example, a liquid solution, such as an effective amount of the composition dissolved in diluents, suspensions in an appropriate liquid, or suitable emulsions. An oral formulation could also be delivered in tablet form, and could include excipients, colorants, diluents, buffering agents, moistening agents, preservatives, flavoring agents, and pharmacologically compatible excipients. A topical formulation could include compounds to enhance absorption or penetration of the active ingredient through the skin or tissue or other affected areas, such as dimethylsulfoxide and related analogs. The pharmaceutical composition could also be delivered topically using a transdermal device, such as a patch or pump, which could include the composition in a suitable solvent system with an adhesive system, such as an acrylic emulsion, and a polyester patch. Compositions could be delivered via eye drops or other topical eye delivery method. Compositions may be delivered intraocularly, anywhere in the eye including, for example, the vitreous cavity, the anterior chamber, etc. Compositions may be delivered subretinally. Compositions may be delivered intravitreally as is commonly done with intravitreal injections. Compositions may be delivered by injecting into the suprachoroidal space. Compositions may be delivered periocularly (e.g. to the tissue around the eyeball (globe) but within the bony orbit). Compositions may be delivered via intraocular implant. In intraocular implant delivery, devices containing compositions of the present invention are surgically implanted (e.g. within the vitreous cavity), and the drug is released into the eye (e.g. at a predetermined rate). Compositions may be delivered via transscleral drug delivery using a device sutured or placed next to the globe that would slowly elute the drug, which would then diffuse into the eye.

As those skilled in the art would recognize, depending on the chosen route of administration (e.g. eye drops, injection, etc.), the composition form is determined. In general, it is preferred to use a unit dosage form of the active agent (e.g., flubendazole) in order to achieve an easy and accurate administration of the active pharmaceutical compound. In general, the therapeutically effective pharmaceutical compound is present in such a dosage form at a concentration level ranging from about 0.5% to about 99% by weight of the total composition: i.e., in an amount sufficient to provide the desired unit dose. In some embodiments, the pharmaceutical composition may be administered in single or multiple doses. The particular route of administration and the dosage regimen will be determined by one of skill in keeping with the condition of the individual to be treated and said individual's response to the treatment. In some embodiments, a composition in a unit dosage form for administration to a subject, comprises a pharmaceutical compound and one or more nontoxic pharmaceutically acceptable carriers, adjuvants or vehicles. The amount of the active ingredient that may be combined with such materials to produce a single dosage form will vary depending upon various factors, as indicated above. A variety of materials can be used as carriers, adjuvants and vehicles in the composition of the invention, as available in the pharmaceutical art. Injectable preparations, such as oleaginous solutions, suspensions or emulsions, may be formulated as known in the art, using suitable dispersing or wetting agents and suspending agents, as needed.

### EXPERIMENTAL

### Example 1

Age-related macular degeneration (AMD), the leading cause of irreversible blindness in the developed world, is marked histologically by the accumulation of lipid-rich deposits in and around the retinal pigment epithelium (RPE). Intracellular lipid-rich accumulations are termed lipofuscin while extracellular accumulations are termed drusen [Refs. 1,2; incorporated by reference in their entireties]. The RPE is a polarized monolayer, facing a fenestrated capillary bed termed the choroid basolaterally and a photoreceptor cell layer apically. The RPE is a high-volume consumer of lipid via uptake of lipoprotein particles from the choroidal circulation and daily ingestion of lipid-rich outer segments (OS) from photoreceptor cells. The RPE is also a prolific lipid secretor, with lipoprotein particles directed apically providing lipid for photoreceptor OS synthesis and lipoprotein particles directed basolaterally sending unneeded lipid through Bruch's membrane to the choroid [Ref. 3; incorporated by reference in its entirety]. The accumulation of intracellular lipofuscin in AMD is likely the result of inefficient breakdown of phagocytosed OS [Ref. 4; incorporated by reference in its entirety], while the accumulation of extracellular drusen likely derive from lipoprotein particles secreted by the RPE and trapped in the underlying basement membrane [Ref 3; incorporated by reference in its entirety]. Improving the RPE's capacity for lipid handling may alleviate each of these histologic hallmarks of AMD.

Autophagy is a major cellular mechanism for degrading both molecules and organelles. A de novo double-membrane autophagosome engulfs target cargo and then fuses with the lysosome to promote degradation of the engulfed content. Autophagy has been implicated in degradation of insoluble pathologic aggregates in neurodegenerative diseases [Ref 5; incorporated by reference in its entirety] and intracellular lipid droplets in adipocytes and hepatocytes [Ref. 6; incorporated by reference in its entirety]. Thus, autophagy activation in the RPE may improve the clearance of insoluble lipofuscin while promoting degradation of the daily lipid load faced by RPE during OS phagocytosis and lipoprotein particle uptake. Efficient degradation of ingested lipids may, in 80 turn, decrease secretion of drusen-inducing lipoprotein particles. Further, breakdown of fatty acids may induce ketone body (KB) production by the RPE. In turn, KB secretion by the RPE, which is almost exclusively apically directed towards photoreceptors, has been shown to provide photoreceptors with an alternate fuel source and may promote photoreceptor survival under stress [Refs. 7,8; incorporated by reference in their entireties]. Thus, autophagy activation has multiple theoretical mechanisms for alleviating AMD phenotypes [Refs. 9,10; incorporated by reference in their entireties].

While hundreds of small molecule and protein targets for autophagy induction have been published [Refs. 11-33; incorporated by reference in their entireties], it has been shown that small molecule inducers of autophagy in one cell type often do not induce autophagy in other cell types [Ref. 12; incorporated by reference in its entirety]. Almost none of the hits from prior autophagy inducer screens have been tested for efficacy and toxicity in RPE. Further, most of the autophagy inducers tested in RPE directly inhibit mTOR or its immediate upstream kinases. Strong mTOR inhibition may disrupt RPE phagocytosis and has failed in a randomized-controlled trial for advanced dry AMD [Refs. 34-36; incorporated by reference in their entireties].

Non-primate models that replicate the features of dry macular degeneration are not available. While some genetic mouse models simulate some features of drusen, none closely recapitulate human drusen morphology and composition [Refs. 37-39; incorporated by reference in their entireties]. Additionally, the structure and composition of lipoprotein particles that underpin human drusen development are markedly different in mice [Ref. 40; incorporated by reference in its entirety]. To complement shortcomings in mouse models, primary human RPE culture models of lipoprotein secretion and drusen formation have been established [Refs. 39,41,42; incorporated by reference in their entireties].

Experiments were conducted during development of embodiments herein to screen small molecule activators of autophagy in primary human RPE culture that are not known to directly target mTOR or its proximal upstream kinases. One of these activators, an FDA-approved anti-helminthic called flubendazole (FLBZ), promotes degradation rather than secretion of ingested lipids, leading to production of photoreceptor-protective KBs while decreasing extracellular drusen-forming lipoprotein particles. FLBZ also decreases the burden of lipofuscin accumulation while alleviating lipofuscin-induced senescence and tight junction disruption.

### Methods:

Primary hfRPE and ARPE-19 Culture

Human fetal eyes were obtained from Advanced Bioscience Resources (ABR, Alameda, California) and cultured [Ref. 43; incorporated by reference in its entirety]. ARPE-19 cells were cultured at passage 19 [Ref. 51; incorporated by reference in its entirety].

### Assaying for Autophagy

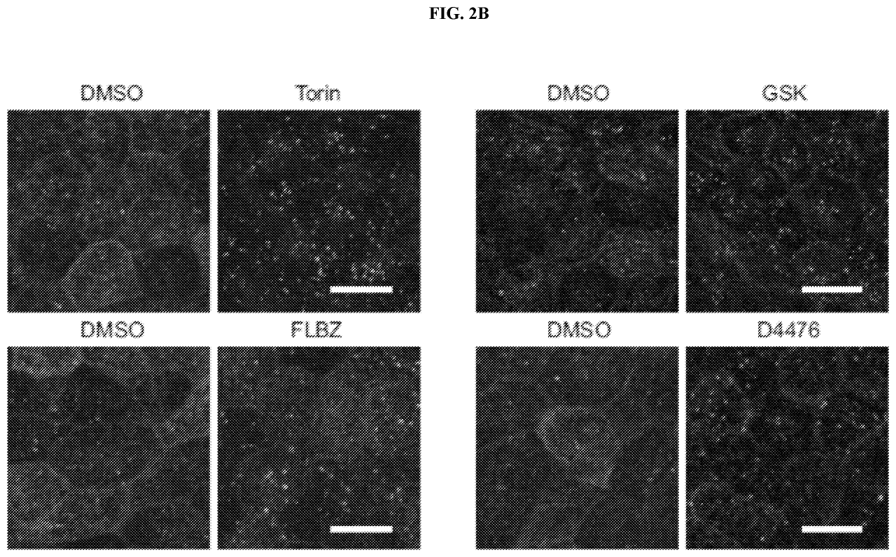

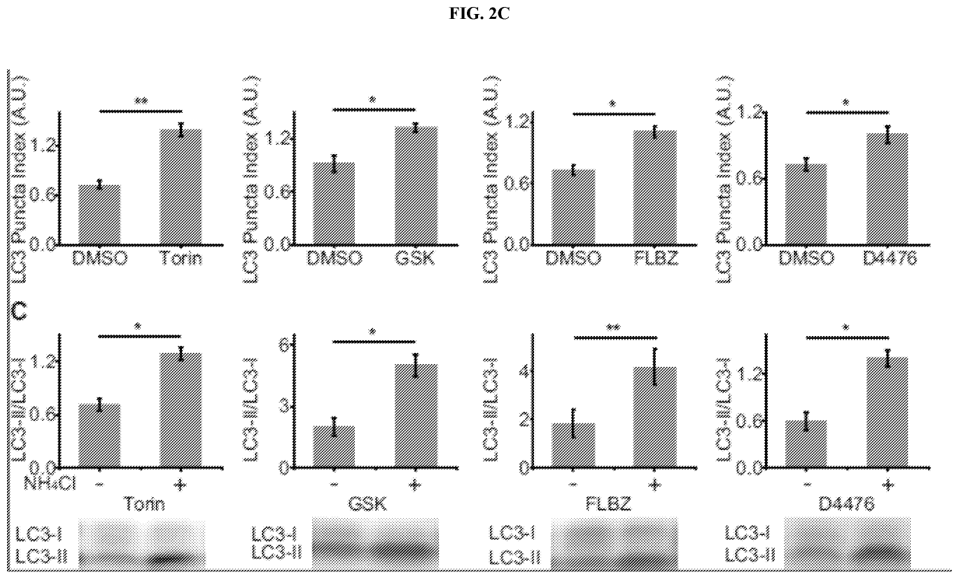

Cell cultures were exposed to each putative autophagy inducer at a concentration indicated in Table 1 for 24 hours, lysed with 36 μL of SDS sample buffer with β-mercaptoethanol, with 15 μg of lysate loaded on a 4-15% gradient gel followed by transfer to a PVDF membrane. Blots were incubated with 1:1000 of LC3A/B antibody (Cell Signaling Technology; #4108s, RRID:AB_2137703) overnight. Quantifying LC3-II/LC3-I ratio is a well-accepted method for det 306 ermining autophagy induction [Ref. 44; incorporated by reference in its entirety], and this ratio was quantified in a non-saturated, linear range using the Azure c500 Imaging System (Azure Biosystems, Dublin, CA, USA) and a combination of AzureSpot and ImageJ software. The data in FIG. 2A are not normalized, whereas the data in FIGS. 2b and 1c are normalized to the average combined value of the vehicle and drug groups within each experimental repeat. The extreme pigmentation of human fetal cultures, combined with the destruction of the LC3 epitope with melanin bleaching protocols, makes detection of LC3 by microscopy in hfRPE difficult. A range of LC3 staining and puncta quantification protocols specifically adapted to pigmented hfRPE culture were employed [Ref. 43; incorporated by reference in its entirety]. At least five fields of view were randomly chosen and analyzed per experimental replicate for LC3 immunocytochemistry.

TABLE 1
Putative Autophagy Inducers Selected for
Study in Primary Human RPE Cultures

| | | Concentration | | |
|---|---|---|---|---|
| | | Tested in | | |
| Name | Abbreviation | This Study | | Citation |
| GSK 1059615 | GSK | 10 | μM | [85] |
| Torin1 | Torin | 1 | μM | [86] |
| D4476 | | 10 | μM | [19, 87] |
| Flubendazole | FLBZ | 12 | μM | [11] |
| Amiodarone | | 10 | μM | [88] |
| GW7647 | | 1 | μM | [13] |
| JNJ-47965567 | JNJ | 10 | μM | [32] |
| 2-Acetyl-5-tetrahydroxybutyl | THI | 10 | μM | [20] |
| Imidazole | | | | |
| Ac-Calpastatin | | 10 | μM | [89] |
| AZ-10606120 | | 10 | μM | [32] |
| Bortezomib | | 50 | nM | [90] |
| BRD5631 | | 10 | μM | [29] |
| Carbamazepine | | 10 | μM | [91] |
| Clonidine | | 10 | μM | [91] |
| Entinostat | MS-275 | 10 | μM | [92, 93] |
| Erlotinib | | 10 | μM | [94] |
| Fasudil | HA-1077 | 10 | μM | [95] |
| Fenofibrate | | 200 | μM | [96] |
| Fluphenazine | | 10 | μM | [97] |
| K604 | | 10 | μM | [26] |
| Loperamide | | 4 | μM | [22] |
| Metformin | | 1 | mM | [27] |
| ML246 | Metarrestin | 10 | μM | [18] |
| Mocetinostat | | 10 | μM | [92, 93] |
| Nilotinib | | 10 | μM | [98] |
| Nilvadipine | | 10 | μM | [99] |
| Oxaprozin | | 10 | μM | [30] |
| Rilmenidine | | 10 | μM | [100] |
| Saroglitazar | | 10 | μM | [101, 102] |
| Sertraline | | 10 | μM | [103] |
| Spermidine | | 10 | μM | [25] |
| Trifluoperazine | | 1 | μM | [22] |
| (±)-Verapamil | | 10 | μM | [27] |

### TEER and Cell Death Assays

TEER and cell death were measured [Ref 51; incorporated by reference in its entirety]. For cell death assays, maximum possible LDH release per Transwell was measured immediately after 2 μL of the final experimental supernate was taken. The Transwell and supernate was then treated with 0.2% Triton X-100 for 15 minutes at 37° C. followed by collection of an additional 2 μL of supernate. Each experimental LDH release value was first normalized to total LDH release from the vehicle condition. Additionally, all TEER and LDH release measurements were normalized to the vehicle group at the zero-hour timepoint.

### Phagocytosis Assays

Phagocytosis assays using bovine outer segments was performed using the “pulse-only” method [Ref 43; incorporated by reference in its entirety]. Values at the zero-hour timepoint were used for normalization.

### Measuring APOE Secretion and Ketogenesis

After 24 hours of exposure to drugs, supernates were collected and subjected to western blotting for APOE detection and a fluorometric assay for β-HB detection [Ref 51; incorporated by reference in its entirety]. Twenty microliters of apical and basolateral supernates for APOE were mixed with SDS sample buffer and blotted with antibody at a dilution of 1:2000. APOE and β-HB values were normalized to the average value of the vehicle and experimental group within each experimental repeat.

### Undigested Autofluorescent Material

UAM accumulation and quantification as well as assays on senescence were carried out [Ref. 51; incorporated by reference in its entirety]. UAM granule size was measured with Leica LAS X software using the length of the long axis of the granule ellipse.

### Statistical Analysis

Means were compared using paired or unpaired two-tailed Student's t-test, as appropriate. All error bars represent standard error of the mean unless otherwise specified. For APOE secretion experiments, there were non-balanced technical replicates between experiments and within treatment groups of a given experimental repeat. To ensure our normalization scheme did not lead to bias, the magnitude and significance of findings was confirmed using mixed effects modeling in R [Ref. 84; incorporated by reference in its entirety]. All results from this analysis were concordant with values reported in this study.

### Results:

### Identifying Autophagy Inducers in RPE

Over 30 putative autophagy inducers were selected that are not known to directly inhibit mTOR or its immediate upstream kinases and tested these inducers in a primary human fetal RPE (hfRPE) culture system [Ref 43; incorporated by reference in its entirety]. Nearly all of the autophagy inducers chosen were FDA115 approved compounds or have a clearly defined protein target under pharmacologic development. Previous literature suggested all compounds should induce autophagy at a low μM concentration, with the exception of fenofibrate and metformin, which have high serum concentrations at clinically relevant doses. Compounds with highly toxic mechanisms (e.g. alkylating agents) were excluded from testing, and within a pharmacologic class, no more than two compounds were tested (Table 1).

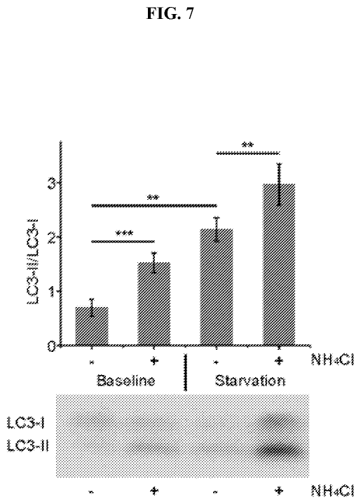

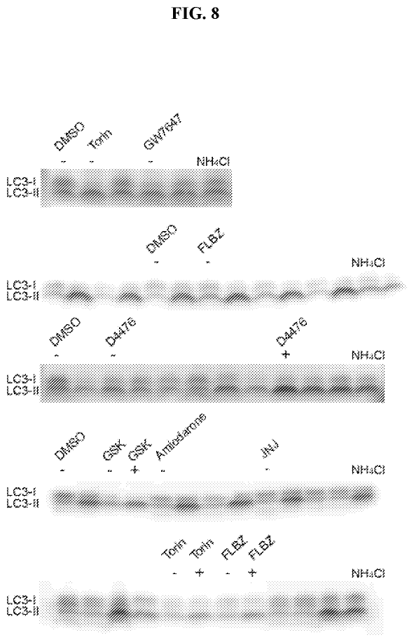

To test for autophagy induction, lipidation of the core autophagy protein LC3 was measured by mobility shift on Western blots 24 hours after each compound was added to hfRPE cultures [Ref. 44; incorporated by reference in its entirety]. It was confirmed that primary RPE cultures upregulate autophagy in response to classical inducers, including mTOR inhibition using the mTOR-specific inhibitor Torin1 (Torin, FIG. 2a) and the upstream pan-phosphoinositide-125 3-kinase and mTOR dual inhibitor GSK1059615 (GSK, FIG. 2a) as well as amino acid/serum starvation (FIG. 7). Of the more than 30 putative autophagy inducers tested, only five produced an increased ratio of LC3-II/LC3-I, consistent with increased autophagy (FIG. 2a).

Autophagy induction was confirmed by assaying the formation of LC3-positive puncta (autophagosomes) by immunocytochemistry [Ref. 44; incorporated by reference in its entirety]. Puncta formation was quantified in automated/unbiased fashion using a customized macro in the Fiji/ImageJ platform [Ref. 43; incorporated by reference in its entirety]. Besides Torin and GSK, only two compounds, D4476, a casein kinase 1 inhibitor, and FLBZ, an FDA-approved anti-helminthic, also induced autophagy by this second assay (FIG. 2b).

Increases in both LC3 lipidation and autophagosome formation could result from downstream blockade of autophagy at the lysosome. To confirm that hits were genuine inducers of RPE autophagy flux, the last step of autophagy flux, lysosomal degradation, was disrupted through alkalinization of the lysosome with ammonium chloride, a well-accepted method for confirming autophagy flux [Ref 44; incorporated by reference in its entirety]. As expected for elevated autophagy flux rather than downstream autophagy blockade, levels of lipidated LC3 rose for all small molecule autophagy inducers (FIG. 2c) and for amino acid/serum deprivation (FIG. 7).

### Safety of Autophagy Inducers

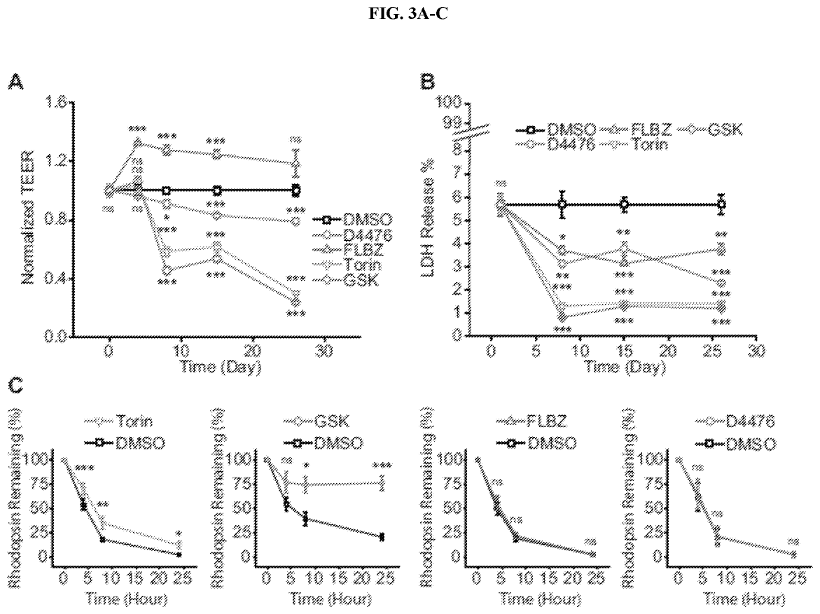

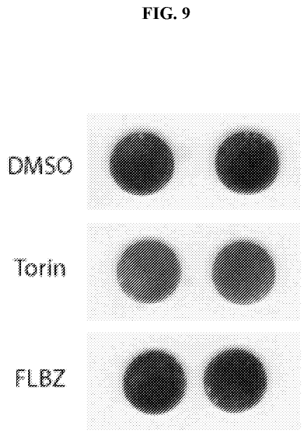

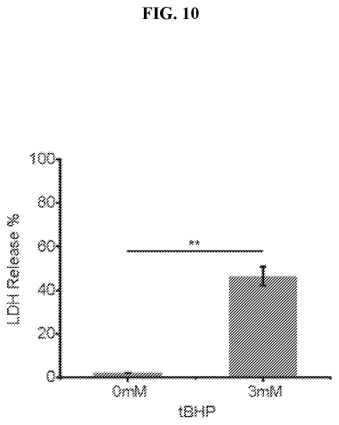

To evaluate the safety of D4476 and FLBZ, trans-epithelial electrical resistance (TEER) was measured, a measure of tight-junction integrity, in RPE cultures after prolonged exposure to each compound. As well-formed tight-junctions require myriad cell processes to be optimally coordinated, assessing TEER provides an easily measurable, ongoing, and noninvasive marker for general RPE cell health [Ref. 45; incorporated by reference in its entirety]. While both mTOR inhibitors, Torin and GSK, dramatically reduced TEER, D4476 had a more modest impact and FLBZ had no negative effect on TEER (FIG. 3a). Reduced RPE pigmentation is associated with increased susceptibility to oxidative insults [Ref. 46; incorporated by reference in its entirety], and in contrast to cultures treated with repeated doses of FLBZ or vehicle for at least 20 days, Torin reduced RPE depigmentation (FIG. 9). Lactate dehydrogenase (LDH) release, a combined marker of necrotic and late-stage apoptotic cell death [Refs. 47,48; incorporated by reference in their entireties], was reduced compared to vehicle for all four verified RPE autophagy inducers (FIG. 3b). Exposure of RPE cultures to the oxidant tert-butyl hydroperoxide confirmed the ability of the assay to detect cell death (FIG. 10).

Daily OS phagocytic uptake and degradation is a core RPE function necessary for retinal function [Ref. 49; incorporated by reference in its entirety]. A previous study suggested that induction of autophagy in the RPE leads to impaired OS degradation after initial phagocytic internalization since autophagy and RPE phagocytosis share overlapping protein machinery [Ref. 28; incorporated by reference in its entirety]. Experiments conducted during development of embodiments herein demonstrated that autophagy induction with mTOR inhibitors did indeed impair breakdown of OS, D4476 and FLBZ had no effect on OS degradative capacity (FIG. 3c).

### Effects of Autophagy Inducers on the Balance Between Lipid Degradation and Lipid Secretion

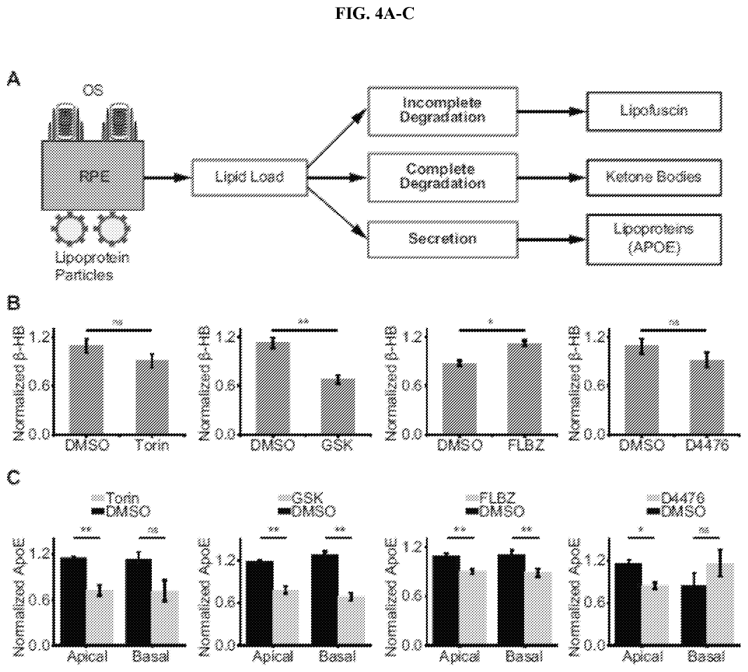

The RPE handles an enormous lipid burden on a daily basis, including OS ingestion from its apical side and lipoprotein particle absorption from its basolateral side. Rather than storing excess lipid, the RPE may choose to degrade or secrete surplus lipids (FIG. 4a). A marker of lipid degradation is the production of KBs, which are secreted by the RPE apically and serve as an energy source for photoreceptors [Refs. 7,8; incorporated by reference in their entireties]. A marker of lipid secretion is the production of the lipoprotein APOE, which is a major component of drusen [Ref. 50; incorporated by reference in its entirety]. It was contemplated that autophagy inducers promote ketogenesis through degradation of ingested lipids, reducing the stimulus for secretion of drusen-promoting APOE. In primary RPE cultures, only FLBZ both increased production of the major KB, β-hydroxybutyrate (β-HB), and reduced the apical and basolateral secretion of APOE, indicating that FLBZ-mediated autophagy induction changes the lipid-handling profile of RPE away from drusen promotion (FIGS. 4b-c).

### Effects of Autophagy Inducers on Lipofuscin-Like Accumulation

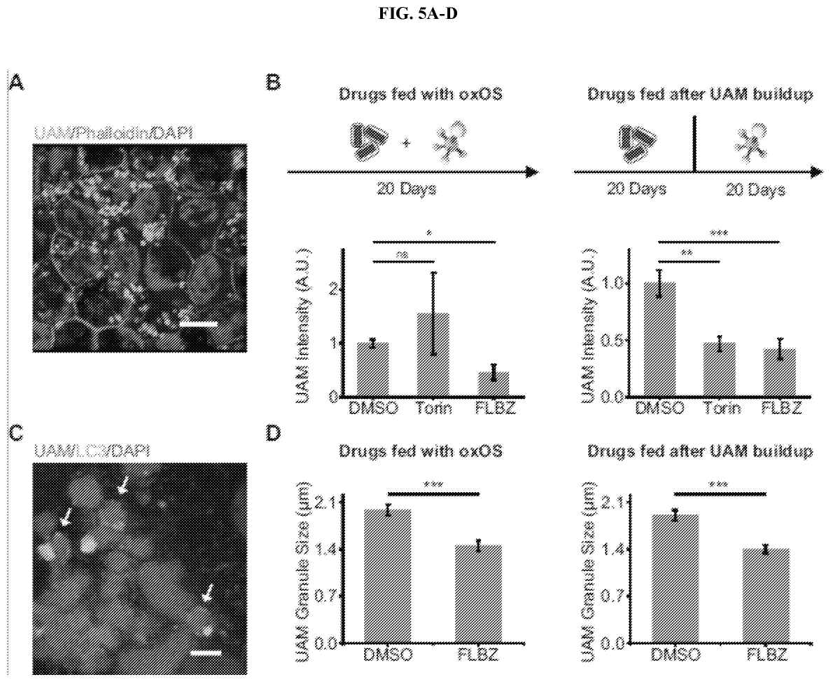

A model of lipofuscin-like material accretion through repeated feeding of photo-oxidized OS (oxOS) to primary hfRPE culture was developed and extensively characterized [Ref. 51; incorporated by reference in its entirety]. Twenty-plus oxOS feedings over the course of a month results in a significant and stable autofluorescent granule burden (FIG. 5a). With time, these granules, termed undigestible autofluorescent material (UAM), resemble the size and emission spectrum of lipofuscin. Like lipofuscin, UAM stain with Nile Red, a marker of neutral lipids, and frequently combine with melanosomes to form melanolipofuscin granules [Ref. 51; incorporated by reference in its entirety]. The concurrent feeding of oxOS and FLBZ at 12 μM over the course of a month resulted in significantly less UAM accumulation compared to feedings of oxOS plus vehicle (FIG. 5b).

Since FLBZ has no effects on phagocytosis efficiency (FIG. 3c), this reduction in UAM accumulation was not due to less uptake of oxOS in the FLBZ group. LC3 colocalization with UAM granules was also observed, supporting a role of autophagy in clearing UAM (FIG. 5c). Remaining UAM granules in FLBZ-treated cultures were smaller than untreated cultures (FIG. 5d) In contrast, treatment with Torin at 1 μM over the course of a month resulted in more UAM accumulation (FIG. 5b), possibly due to inhibitory effects of Torin on phagocytic degradation (FIG. 3c).

To determine whether FLBZ could reduce UAM after it had accumulated, cultures were fed with oxOS twenty times over the course of a month, then extensively washed off OS from the cultures, and subsequently treated with twenty repeated drug feedings over the course of an additional month. FLBZ led to significantly lower levels of UAM and smaller granule size, confirming that UAM is compactable and/or removable even after its accrual in the RPE (FIG. 5b, d).

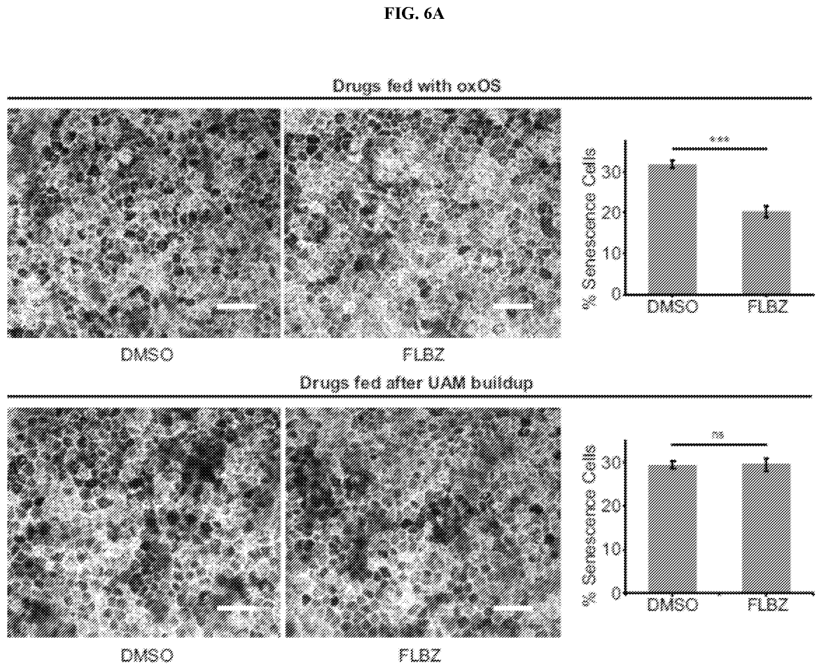

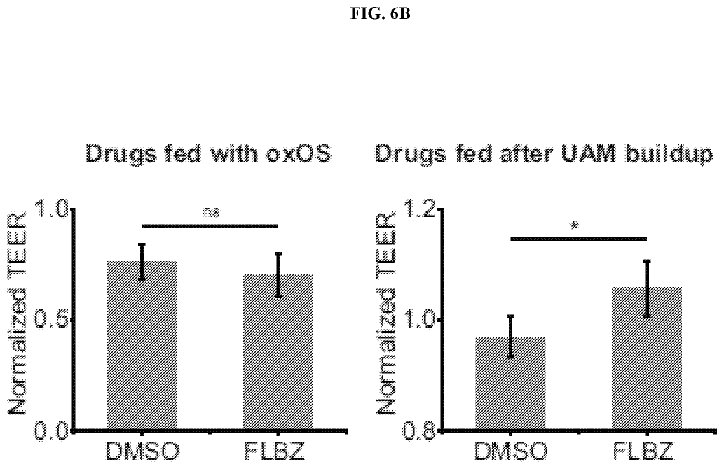

Cultures with a high UAM burden demonstrate significant senescence [Refs. 51,52; incorporated by reference in their entireties], which in the RPE can contribute to the para-inflammatory state characteristic of AMD [Ref. 53; incorporated by reference in its entirety]. Cofeeding cultures with oxOS and FLBZ resulted in less senescence compared to feeding of oxOS plus vehicle (FIG. 6a). When cultures were treated with FLBZ after UAM had already accumulated, cell senescence was not decreased, indicating that established RPE senescence may be difficult to reverse (FIG. 6a). Nevertheless, FLBZ still improved cell health, as assessed by tight-junction integrity, in cultures with already established UAM (FIG. 6b).

Dry AMD may be a disease of perturbed lipid homeostasis, characterized by extracellular deposition of lipid-rich drusen. The accumulation of lipid-rich intracellular lipofuscin may also be linked to AMD and Stargardt macular dystrophy [Refs. 54,55; incorporated by reference in their entireties]. Efficient degradation of ingested lipid by the RPE produces KBs that are secreted and then utilized by photoreceptors for metabolism. RPE ketogenesis may promote survival of metabolically-stressed photoreceptors deprived of their primary energy source, glucose [Refs. 7,8; incorporated by reference in their entireties]. For all these reasons, improving RPE lipid handling is an attractive therapeutic approach for the treatment of dry AMD.

Autophagy induction, which promotes lipid degradation and turnover of otherwise undigestible cellular constituents, has the potential to favorably impact RPE lipid homeostasis. However, experiments conducted during development of embodiments herein demonstrate that mTOR inhibition, the most common pathway for inducing autophagy, disrupts multiple RPE-specific functions. Furthermore, the mTOR inhibitor, sirolimus, failed to demonstrate therapeutic benefit in a randomized controlled trial of advanced dry AMD [Ref. 36; incorporated by reference in its entirety].

Of more than 30 putative autophagy inducers tested, only two, D4476 and FLBZ, reliably induced autophagy in primary human RPE cultures.

In the model of RPE lipid handling (FIG. 4a), the RPE ingests OS and lipoprotein particles as part of a daily lipid challenge. If OS are incompletely degraded, lipofuscin develops. Lipid that is fully degraded in the lysosome transits the endoplasmic reticulum and is packaged as lipid droplets. The lipid droplets that form after an RPE lipid challenge, however, dissipate quickly [Ref 64; incorporated by reference in its entirety], in contrast to the longer-lived lipid droplets of adipocytes or hepatocytes. Further, the accumulation of bloated lipid vacuoles, a feature of many age-related diseases including atherosclerosis [Ref. 65; incorporated by reference in its entirety], cardiomyopathy [Ref 66; incorporated by reference in its entirety], liver disease [Ref. 65; incorporated by reference in its entirety], and neurodegeneration [Refs. 67,68: incorporated by reference in their entireties], is not a feature of RPE degeneration in macular degeneration. Thus, the RPE's large daily lipid load is actively degraded or secreted rather than stored long-term. Tipping the balance towards degradation may provide photoreceptors with KBs, an alternative fuel source, and decrease the amount of secreted lipoprotein that contributes to drusen formation. It was found that FLBZ altered the balance between degradation vs. secretion of lipid in primary human RPE culture. By reducing lipid secretion, FLBZ has the potential to work synergistically in dry AMD with pharmacologic programs aimed at clearing already deposited lipid/drusen in Bruch's membrane [Refs. 69,70; incorporated by reference in its entirety].

Existing pharmaceutical approaches to lipofuscin reduction have focused on disrupting the retina's visual cycle, which produces the retinoids that contribute to lipofuscin accumulation. However, visual cycle modulators have failed to date in human clinical trials, likely because the visual cycle is so integral to visual function [Ref 71; incorporated by reference in its entirety]. Avoiding the disadvantages of visual cycle modulation, it was determined that autophagy induction both prevented and reduced the accumulation of lipofuscin-like material (i.e. UAM [Ref. 51; incorporated by reference in its entirety]) in primary RPE culture. Autophagy may clear lipofuscin both by wholesale engulfment of granules in a process akin to autophagic engulfment of lysosomes [Ref. 72; incorporated by reference in its entirety] and by the natural upregulation of lysosomal capacity that accompanies autophagy induction [Refs. 73,74; incorporated by reference in their entireties].

Under conditions where FLBZ prevented UAM accumulation, the drug also reduced senescence associated with UAM. Reduced senescence may decrease AMD-associated inflammation and neovascularization [Refs. 75,76; incorporated by reference in their entireties], and drugs that specifically eliminate senescent cells are in early stage clinical development for macular degeneration [Refs. 77,78; incorporated by reference in their entireties]. Consistent with reports on the difficulty of reversing senescence [Ref 79; incorporated by reference in its entirety], FLBZ did not reduce senescence in cultures where UAM had already accumulated. Nevertheless, delaying FLBZ treatment until after UAM had fully accumulated still reduced UAM burden, compacted remaining UAM granules, and improved cell health, as assessed by tight-junction integrity. UAM granules slowly compact over 12 months in culture; the acceleration of this compaction process by autophagy may diminish reactivity and toxicity of lipofuscin-like granules [Ref. 51; incorporated by reference in its entirety].

### Example 2

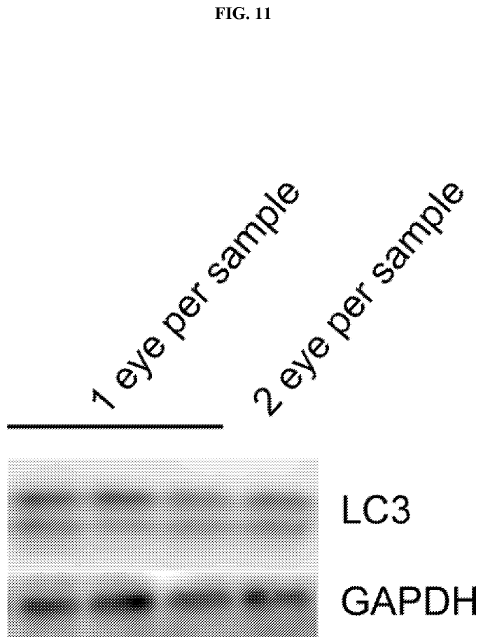

Experiments were conducted during development of embodiments herein to assay for autophagy in the mouse eye. Experiments were conducted to measure autophagy by giving an intraperitoneal injection of leupeptin, followed by assaying for LC3 puncta in an RPE flatmount of a GFP-LC3 mouse. Background from GFP-LC3 debris deposited on the RPE when the retina was peeled off for the flat mount prevented signal detection. A biochemical assay was then employed for autophagy induction, measuring an increase in the lipidation of the autophagy core protein LC3 (lipidated LC3=LC3-II, unlipidated LC3=LC3-I, so higher LC3-II/LC3-I ratio indicates autophagy induction). Sufficient signal as obtained. The protocol was optimized to determine the minimum number of mouse eyes needed to detect LC3 bands in the lysate and found that 1 eye worked as well as 2 pooled eyes, allowing an increase in n using the same number of mice (FIG. 11). Harvesting techniques were also optimized for collecting RPE from mouse eyes.

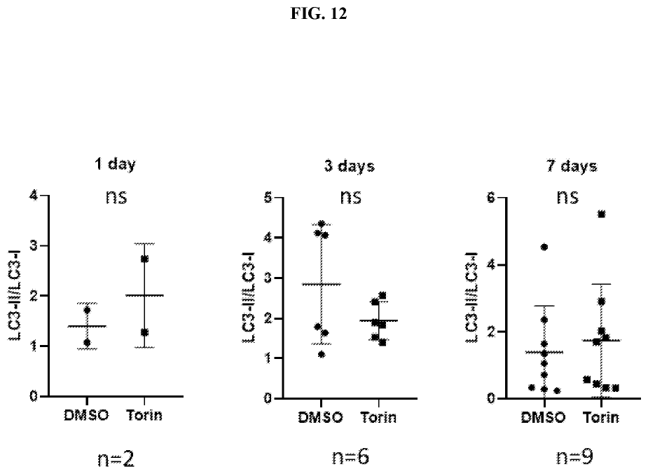

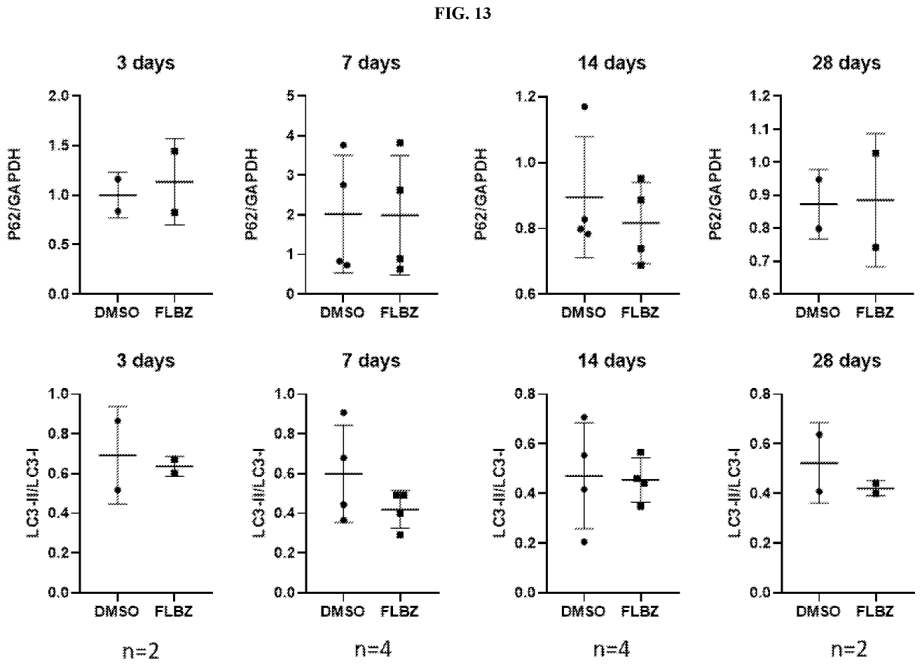

Experiments were conducted during development of embodiments herein to establish a positive control for autophagy induction. The direct mTOR inhibitor Torin1 was selected over rapamycin/sirolimus as the positive control, based on the capacity of Torin1 to induce autophagy in primary culture more strongly and more consistently than rapamycin. The experimental set-up involved injecting one eye with Torin and the other eye with the DMSO vehicle. Autophagy induction was measured at 4 different concentrations of Torin (500 nm, 5 um, 50 um, 500 um) and at 5 different timepoints after injection (1 day, 3 days, 7 days, 14 days, 28 days). No concentration or time after injection provided convincing evidence for replicable autophagy induction using Torin (FIG. 12). Nevertheless, the ability of intravitreal injections of FLBZ to induce autophagy in the RPE was assessed. In preparation for injecting FLBZ intravitreally into mice, solubility assays were performed and the maximum amount of FLBZ that can be injected without it crashing out of solution in the vitreous was determined. Torin was kept in a 1 mM stock solution, dissolved in DMSO, and did not crash out when mixed 1:1 with PBS (simulating the vitreous). In contrast, a 3 mM stock solution of flubendazole in DMSO crashed out when mixed with PBS unless at a dilution of 1:20 or more. This helped inform maximal concentrations of flubendazole that could be injected intravitreally. Four different concentrations were injected, up to the maximum solubility of FLBZ, and autophagy induction was assayed over a timeframe ranging from 1 week to 4 weeks after injection. Like Torin, no convincing evidence for autophagy induction was observed in the RPE upon intravitreal injection (FIG. 13).

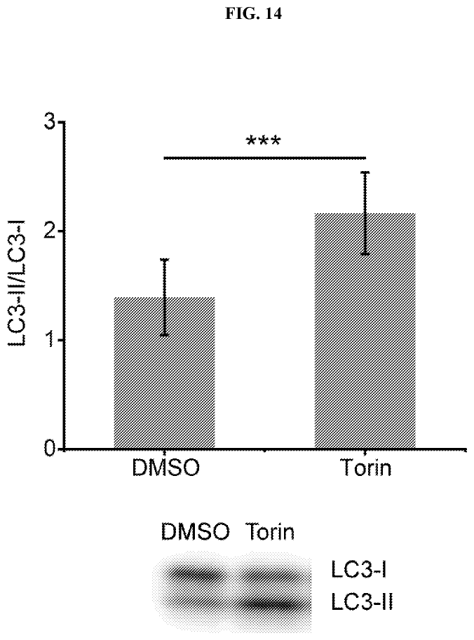

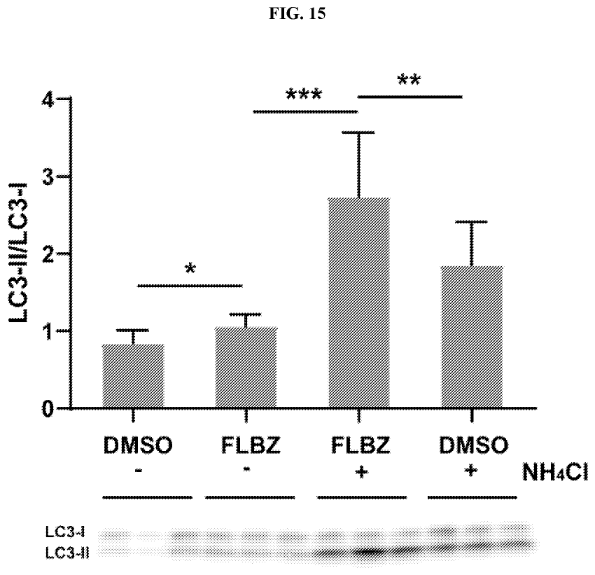

Given the inconsistent results with detecting autophagy induction using intravitreal injection of the positive control, Torin, experiments were conducted to determine whether the lack of responsiveness to intravitreal injections was a drug access issue or whether it was a species or animal vs. culture difference. Additional experiments utilized freshly isolated mouse eyecups with the retina removed to determine whether Torin (and FLBZ) induce autophagy in this context. Indeed, Torin at 1-5 uM induces autophagy well in this context (FIG. 14) and FLBZ at 12-60 uM also induces autophagy well (FIG. 15). The use of ammonium chloride in these experiments blocks lysosomal degradation. Drugs that truly increase autophagy flux will trigger a higher LC3-II/LC3-I ratio with ammonium chloride than without ammonium chloride, and this was demonstrated in our vehicle and FLBZ treated eyecups (FIG. 15).

Experiments conducted during development of embodiments herein demonstrate that that the limited solubility of FLBZ in DMSO, coupled with the small size of the mouse vitreous cavity made this system difficult for taking accurate measurements. If a model (e.g., rat, rabbit, human) with a vitreous cavity were used, less DMSO needs to be delivered with the drug, giving the drug a greater likelihood of solubilizing in the aqueous vitreous environment.

### Example 3

Experiments were conducted during development of embodiments herein to demonstrate that other small molecules (other than flubendazole) that are not direct mTOR inhibitors can induce autophagy in primary human RPE cultures in a non-toxic way. The anti-VEGF receptor drugs, linifanib (ABT-869, anlotinib, patinib, nintendanib (BIBF-112), pazopanib, dasatinib, sorafenib, regorafenib, sunitinib, vandetanib, cabozantinib (XL184) were tested for autophagy induction in primary human RPE cultures.

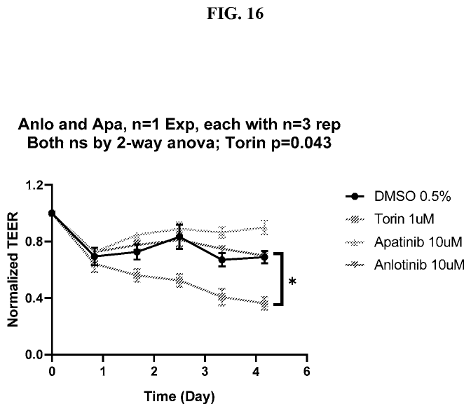

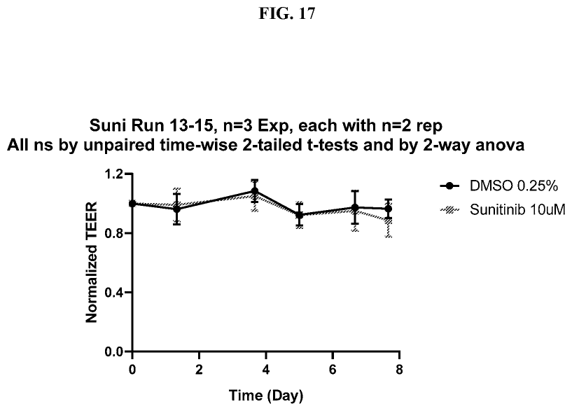

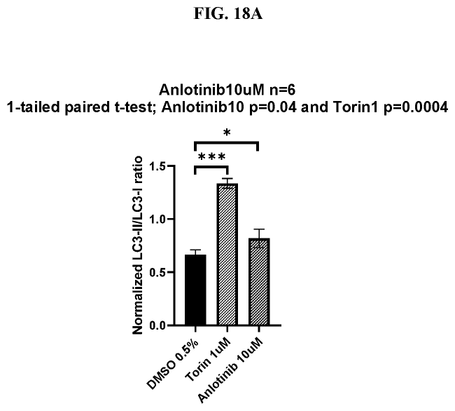

Transepithelial Electrical Resistance (TEER) was tested (FIGS. 16 and 17). TEER is a sensitive measure of toxicity (lower the TEER, the more toxic the drug is). Drugs were added daily and measurements made daily for at least 4-8 days.

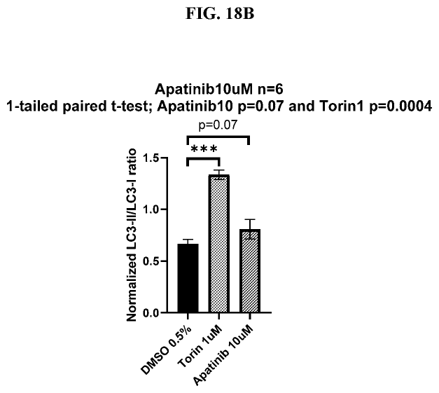

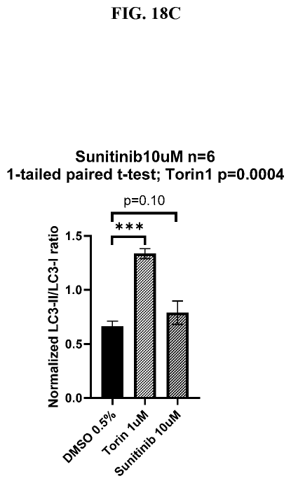

Autophagy induction was measured by LC3-II/LC3-I Ratios on Western Blot (standard method for autophagy induction) (FIG. 18A-C). Drugs were applied to primary human RPE culture for 24 hours.

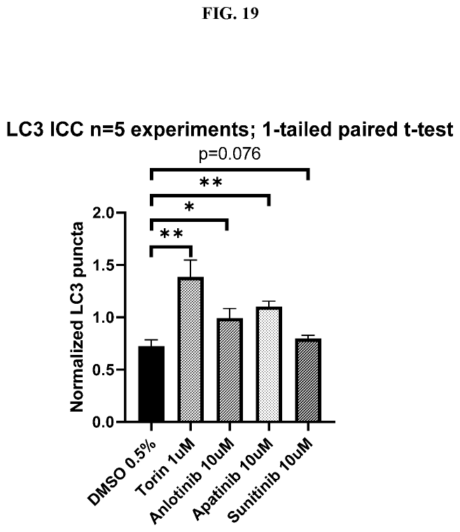

Autophagy induction was measured by LC3 puncta on immunocytochemistry (standard method for autophagy induction) (FIG. 19). Drugs applied to primary human RPE culture for 24 hours.

1. A method of treating or preventing dry age-related macular degeneration (dAMD) in a subject comprising inducing autophagy in the retinal pigment epithelium (RPE) of the subject without direct inhibition of mammalian target of rapamycin (mTOR).

2. The method of claim 1, wherein autophagy is induced in the RPE by administration of a pharmacologic activator of autophagy to the RPE that does not directly inhibit mTOR.

3. The method of claim 2, wherein the pharmacologic activator of autophagy does not bind mTOR.

4. The method of claim 2, wherein the pharmacologic activator of autophagy is administered directly to the eye of the subject.

5. The method of claim 4, wherein the pharmacologic activator is administered intravitreally, subretinally, suprachoroidally, or periocularly.

6. The method of claim 2, wherein the pharmacologic activator of autophagy is administered systemically to the subject.

7. The method of claim 2, wherein the pharmacologic activator of autophagy is flubendazole.

8. The method of claim 1, wherein the subject exhibits one or more risk factors of developing dAMD or exhibits early signs or symptoms of dAMD.

9. A method comprising:
(a) testing a subject for dAMD; and
(b) treating a subject that exhibits signs or symptoms of dAMD with the method of one of claims 1-8.

10. The method of claim 9, wherein testing the subject for dAMD comprises one or more of an eye exam; review of medical history, review of family medical history, examination of the back of the eye, testing for defects in the center of the field of vision, angiography, and optical coherence tomography.

11. The method of claim 9, wherein a subject is determined to be at risk of dAMD and/or exhibits early signs or symptoms of dAMD and the subject is treated to prevent development and/or advancement of dAMD.

11. A pharmaceutical composition comprising (a) a pharmacologic activator of autophagy that does not directly inhibit mammalian target of rapamycin (mTOR) and (b) a pharmaceutically acceptable carrier.

12. The pharmaceutical composition of claim 11, wherein the pharmacologic activator of autophagy is flubendazole.

13. The pharmaceutical composition of claim 11, wherein the pharmaceutical composition is formulated for delivery to the RPE.

14. The pharmaceutical composition of claim 13, wherein the pharmaceutical composition is formulated intravitreally, subretinally, suprachoroidally, or periocularly.
